(19) United States
(12) Patent Application Publication
(10) Pub. No.: US 2004/0057934 A1
(43) Pub. Date: Mar. 25, 2004

(54) Therapeutic agent for treating retroviral infection

(76) Inventor: Hisashi Fujimura, Chitose-shi (JP)

Correspondence Address: CROWELL & MORING LLP, INTELLECTUAL PROPERTY GROUP, P.O. BOX 14300, WASHINGTON, DC, 20044-4300, US

(21) Appl. No.: 10/247,744

(22) Filed: Sep. 20, 2002

(51) Int. Cl. A61K 45/00

(52) U.S. Cl. 424/93.4; 424/780

(57) ABSTRACT

A therapeutic agent for treating diseases caused by retroviruses is provided. The agent acts by inducing secretion of biological anti-microbial peptides. Specifically, the therapeutic agent is intended to treat retroviral infections and contains as an active ingredient fermentation products produced by yeasts and lactic acid bacteria. Among the diseases caused by retrovirus that can be treated with the therapeutic agent are acquired immune deficiency syndrome (AIDS) and adult T-cell leukemia (ATL) caused by human T-cell lymphotropic virus-I (HTLV-I).

### BACKGROUND AND SUMMARY OF THE INVENTION

The present invention relates to a therapeutic agent for treating infectious diseases caused by retroviruses, in particular, acquired immune deficiency syndrome (AIDS) and adult T-cell leukemia (ATL), that acts by enhancing innate immune responses of a living body to promote secretion of biological anti-microbial peptides such as defensin, thereby eliciting anti-viral effects.

Acquired immune deficiency syndrome, or AIDS in short, is brought about by devastation of immune activities as a result of destruction of helper T-cells, a type of cells responsible for immune function, by viral infection.

AIDS is known as a disease that attacks immune system, which is one of fundamental functions required for the sustenance of human life. For this reason, fighting the disease is one of the major challenges that modern medicine faces.

First reported in 1980's, AIDS has become widespread around the globe in an alarmingly short period of time and still keeps spreading. WHO estimates nearly 5,000 to 6,000 people are newly infected each day. The ministry of health in UK even estimates the number amounts to 16,000 people/day.

According to the survey conducted by UNICEF, 40 million children will lose their parents to AIDS by 2010. The situation is particularly serious in East Africa where nearly 40% of all children are said to lose one of their parents before they reach 15 years old because of the lack of necessary treatment and preventive measures against the disease.

Diagnostic techniques needed for diagnosing HIV infection have already been developed and are now in practical use, making diagnosis of the disease possible even in developing countries. Cure for the disease has also been actively developed and it is now possible to delay the onset of the disease in the infected patients. Nevertheless, only a small number of patients in developing countries can afford the expensive medicaments, with most left beyond the reach of benefits of the recent advancement in medicine. On the other hand, researchers in developed countries have yet to understand the precise mechanisms of this disease, which is associated with numerous serious complications. Still, much needs to be done to improve already-existing countermeasures and to develop AIDS vaccines.

AIDS is a disease caused by infection with a retrovirus. The pathogen of AIDS is human immunodeficiency virus, or HIV, an RNA virus belonging to family Retroviridae. HIV often infects lymphocytes and destroys them. The resulting decrease in the number of lymphocytes leads to deficient cellular immunity (acquired) and ultimately to the onset of AIDS.

In patients whose acquired immune functions, which are mainly provided by lymphocytes, are damaged, the role of innate immune responses becomes more significant. Thus, a substance capable of modulating immunity may be deliberately administered to patients suffering from AIDS to promote innate immune responses in their bodies. Such a substance acts to promote protective reactions by stimulating immune responses against viral or microbial infection and can thus serve as an effective therapeutic agent generally applicable to diseases caused by microorganisms or viruses. Such a substance should serve as a clinically effective antiviral agent, especially when it exhibits no toxicity to human body and is safe for administration to human patients.

Japanese Patent Laid-Open Publication No. 2000-125810 (which is incorporated herein by reference in its entirety) discloses a concentrate of a bacterial culture filtrate obtained by co-culturing a particular combination of several different types of beneficial bacteria selected from yeasts and lactic acid bacteria. Aside from its anti-inflammatory activity, the concentrate has activities to modulate and restore immunity. It is concentrated so that it contains amino acids, vitamins, and minerals in a well-balanced manner and exhibits anti-infection activity based on immuno-modulatory/restoring activity.

The anti-infection activity of such concentrate has suggested to the present inventors the possibility of the substance as an effective enhancer of innate immune responses, and thus, as an effective therapeutic agent for treating patients with defective immune functions, in particular for those suffering AIDS. Specifically, the present inventors suspected that if the anti-infection activity of such concentrate was brought about by the stimulation of innate immune responses, then the exhibition of such activity must be accompanied by induction of secretion of defensin, a biologically-produced, anti-microbial peptide responsible for innate immune responses. The peptide then exhibits the antiviral activity.

To examine this hypothesis, the present inventors administered the concentrate to retrovirus-infected mice serving as an animal model of HIV infection and observed the animals for evidences of anti-retroviral activity. As a result, the concentrate has been determined to exhibit strong anti-retroviral activity and has proven to be particularly effective in alleviating acute symptom of retroviral infection.

The present invention has been devised in view of these observations and aims at provision of a therapeutic agent for infectious diseases caused by retroviruses that contains, as an active ingredient, fermentation products produced by yeasts and lactic acid bacteria.

Accordingly, one essential embodiment of the present invention is a therapeutic agent for treating retroviral infection. The therapeutic agent contains a concentrate of a filtrate of a culture solution obtained by co-culturing a particular combination of different types of beneficial bacteria selected from yeasts and lactic acid bacteria.

In a specific example of the therapeutic agent of the present invention, the retroviral infection is acquired immune deficiency syndrome (AIDS) caused by human immunodeficiency virus (HIV).

In another example of the therapeutic agent of the present invention the retroviral infection is adult T-cell leukemia (ATL) caused by human T-cell lymphotropic virus-I (HTLV-I).

Other objects, advantages and novel features of the present invention will become apparent from the following detailed description of the invention when considered in conjunction with the accompanying drawings.

### BRIEF DESCRIPTION OF THE DRAWING

FIG. 1 is a photographic representation showing spleens collected from test mice used in Test 1 of the present invention.

### DETAILED DESCRIPTION OF THE PREFERRED EMBODIMENTS

As previously described, the present invention provides a therapeutic agent for treating retroviral infection that contains, as an active ingredient, fermentation products produced by yeasts and lactic acid bacteria. The therapeutic agent is capable of effectively modulating innate immunity of living bodies and is thereby capable of, for example, inducing secretion of anti-microbial peptides responsible for biological protective functions. In this manner, the therapeutic agent can elicit anti-retroviral activity of the anti-microbial peptides and can thus be used to treat infectious diseases caused by retroviruses.

Examples of the infectious disease caused by retroviruses include acquired immune deficiency syndrome (AIDS) caused by human immunodeficiency virus (HIV) and adult T-cell leukemia (ATL) caused by human T-cell lymphotropic virus-I (HTLV-I).

The therapeutic agent for treating retroviral infection provided in accordance with the present invention has proven to be particularly effective in alleviating acute symptom of retroviral infection. Accordingly, the therapeutic agent of the present invention can be effectively used to alleviate various fulminating symptoms, such as Pneumocystis carini pneumonia and Kaposi's sarcoma, that result from deficient immunity and occur during early stages after onset of AIDS.

The concentrate of the bacterial culture filtrate as an active ingredient of the therapeutic agent for treating retroviral infection for use in the present invention is prepared by co-culturing a particular combination of several different types of beneficial bacteria selected from yeasts and lactobacilli, and in particular, is prepared according to the method described in Japanese Patent Laid-Open Publication No. 2000-125810, whose applicant is YOUNG Co., Ltd. The condensate (referred to as “Young extract,” hereinafter) is marketed by YOUNG Co., Ltd. as a concentrated health beverage. Through an analysis of the ingredients, it was determined that the concentrate contained proteins, phosphorus, iron, calcium, thiamine (vitamin B1), riboflavin (vitamin B 6), and nearly twenty different types of amino acids, including essential amino acids such as threonine, valine, lysine, and leucine, and other amino acids such as glutamic acid, aspartic acid, and arginine.

Examples of yeasts and lactic acid bacteria serving beneficial bacteria for use in the Young extract, the active ingredient of the present invention, are listed in Table 1 below.

TABLE 1

| NO. | BENEFICIAL BACTERIA | |
|---|---|---|
| 1 | Bac. Bulgaricus | No. 1 |
| 2 | Bac. Bulgaricus | No. 2 |
| 3 | Bac. Koernchen | No. 1 |
| 4 | Bac. Koernchen | No. 2 |
| 5 | Bac. Koernchen | No. 3 |
| 6 | Bac. Koernchen | No. 4 |
| 7 | Bac. Acidophillus | No. 1 |
| 8 | Bac. Acidophillus | No. 2 |
| 9 | Bac. Acidophillus | No. 3 |
| 10 | Bac. Acidophillus | No. 4 |
| 11 | Bac. Acidophillus | No. 5 |
| 12 | Bac. Acidophillus | No. 6 |
| 13 | Micrococcus Lactis | No. 1 |
| 14 | Micrococcus Lactis | No. 2 |
| 15 | Micrococcus Lactis | No. 3 |
| 16 | Micrococcus Lactis | No. 4 |
| 17 | Yeast | No. 1 |
| 18 | Yeast | No. 2 |
| 19 | Yeast | No. 3 |
| 20 | Yeast | No. 4 |

As described above, different types of the beneficial bacteria (yeasts and lactic acid bacteria) are used in different combinations. Examples of such combination are shown in Table 2 below. The numbers in Table 2 correspond to the numbers used in Table 1.

TABLE 2

| | |
|---|---|
| Group I | 1 + 3 + 9 + 13 + 17 |
| Group II | 2 + 4 + 10 + 14 + 18 |
| Group III | 7 + 5 + 11 + 15 + 19 |
| Group IV | 8 + 6 + 12 + 16 + 20 |

The Young extracts to serve as the active ingredient of the present invention are produced using a method disclosed in Japanese Patent Laid-Open Publication No. 2000-125810. Specifically, the method involves the following steps.

(1) First Step (Preparation of Special Agar Culture Medium)

Special agar culture medium is prepared by boiling beef and kelp in distilled water to obtain broth and adding the broth to purified natural agar along with salt and a culture solution of the beneficial bacteria.

(2) Second Step (Pure Culture of Beneficial Bacteria)

Different types of the beneficial bacteria are individually inoculated onto the special agar culture medium prepared in the first step. Each culture is then incubated in an incubator for 48 to 50 hours at39° C.

(3) Third Step (Preparation of Special Soybean Milk Culture Medium)

Degreased soybean is boiled in distilled water for 70 to 110 min and is then filtered. Salt, soft brown sugar, and a culture solution of the beneficial bacteria are added to the soybean milk filtrate to obtain special soybean milk culture medium.

(4) Fourth Step (Pure Culture of Beneficial Bacteria in Special Soybean Milk Culture Medium)

Each pure culture of the different types of the beneficial bacteria prepared in the second step is inoculated onto the special soybean milk culture medium prepared in the third step. The culture is subsequently incubated in an incubator for 48 to 50 hours at 39° C.

(5) Fifth Step (Moderate Scale Acclimation Culture of Beneficial Bacteria)

The pure-cultures of beneficial bacteria prepared in the fourth step are divided into different groups, and each group is inoculated onto the special soybean culture medium obtained in the third step and is acclimatized for 48 to 50 hours at 39° C.

(6) Sixth Step (Industrial Scale Co-Culture of Beneficial Bacteria)

After acclimation culturing, each group of the bacteria prepared in the fifth step are inoculated in 13L special soybean milk culture obtained in the third step and are then co-cultured for 100 to 120 hours at 40° C.

(7) Seventh Step (Termination of Culturing and Concentration)

The co-culture solution prepared in the sixth step is heated for 30 min at 100° C. to terminate the growth of bacteria. The solution is filtered to remove bacterial cells and the resulting filtrate is concentrated about 15-fold (by removing 93 to 96% water).

(8) Eighth Step (Aging)

The concentrate obtained in the seventh step is allowed to stand for over 3 months at 17° C. for aging.

The products obtained through the series of steps are placed in sealed vials and are subjected to heat treatment according to legal standards to obtain Young extracts.

By orally administering the Young extracts, secretion of anti-microbial peptides responsible for innate immune responses is facilitated, and as a result, anti-retroviral activity is expressed. Since the safety of the Young extracts as a concentrated health beverage has been previously verified, it may be administered in excess without any adverse effects.

### EXAMPLES

The present invention will now be described in further detail with emphasis on the anti-retroviral activity of the therapeutic agent for retroviral infection provided in accordance with the present invention.

The Young extracts were administered to retrovirus-infected mice, which serve as an animal model of retroviral infection, and the effects were examined.

### Test 1

Test for Determining Effects of Young Extracts on Friend Leukemia Virus

Friend leukemia virus is a retrovirus isolated from Ehrlich-Lettre ascites carcinoma and, when inoculated to sensitive mice, gives rise to erythroblastosis within a time period ranging from a few days to two weeks, causing leukemia accompanied by a swollen spleen. Thus, Friend leukemia virus, which belongs to the same retrovirus family as human immunodeficiency virus (HIV) and human T-cell lymphotropic virus-I (HTLV-I), is typically used to determine anti-viral activity of therapeutic agents of HIV infection such as AZT. The onset of the disease is readily determined by comparing the weights of spleen.

Methods

Friend leukemia virus was inoculated in the abdominal cavity of male BALB/c mice (5 animals per one group) to allow leukemia to develop.

The Young extract was diluted 100-fold with distilled water, and 100 ml of the extract solution was placed in a water supply bottle to allow the mice to ingest the extract solution. The solution was replaced with a fresh supply every other day. The amount of the solution ingested by each mouse was 5 ml/day/mouse on average.

The Young extract was administered over 10 consecutive days starting one day prior to infection with Friend leukemia virus. On day 10, spleen was collected from each animal and the weight was compared to determine the effects.

As a comparative experiment, a group of mice (5 animals) were inoculated with Friend leukemia virus but not administered with the Young extract. In a control group (3 animals), mice were not infected with Friend leukemia virus, nor were they administered with the Young extract.

Results

Weight of each of the collected spleen is shown in Table 3 below.

TABLE 3

| | | Treatment with | Weight of |
|---|---|---|---|
| | Infected virus | Young extract | spleen (g) |
| Test date | Inoculated(day 0) | Administered | Day 10 |
| | | from −1, 0, 1 | |
| | | to day 10 | |
| Mice #1 M | Friend Leukemia | no treatment | 0.998 |
| | virus | | |
| Mice #2 M | Friend Leukemia | no treatment | 0.703 |
| | virus | | |
| Mice #3 M | Friend Leukemia | no treatment | 0.382 |
| | virus | | |
| Mice #4 M | Friend Leukemia | no treatment | 0.722 |
| | virus | | |
| Mice #5 M | Friend Leukemia | no treatment | 0.574 |
| | virus | | |
| Group 1 | Viral infection(+) | no treatment(−) | Mean: 0.676 |
| | | | SD: 0.225 |
| Mice #6 M | Friend Leukemia | treated | 0.608 |
| | virus | | |
| Mice #7 M | Friend Leukemia | treated | 0.351 |
| | virus | | |
| Mice #8 M | Friend Leukemia | treated | 0.432 |
| | virus | | |
| Mice #9 M | Friend Leukemia | treated | 0.319 |
| | virus | | |
| Mice #10 M | Friend Leukemia | treated | 0.315 |
| | virus | | |
| Group 2 | Viral infection(+) | treated(+) | Mean: 0.405 |
| | | | SD: 0.123 |
| Mice #11 M | Not inoculated | no treatment | 0.132 |
| Mice #12 M | Not inoculated | no treatment | 0.168 |
| Mice #13 M | Not inoculated | no treatment | 0.177 |
| Group 3 | No viral infection | no treatment(−) | Mean: 0.159 |
| | (−) | | SD: 0.024 |

Group 1 (mouse #1 to 5): Friend leukemia virus (+); given distilled water.

Group 2 (mouse #6 to 10): Friend leukemia virus (+); given Young extract.

Group 3 (mouse #11 to 13): Friend leukemia virus (−); given distilled water.

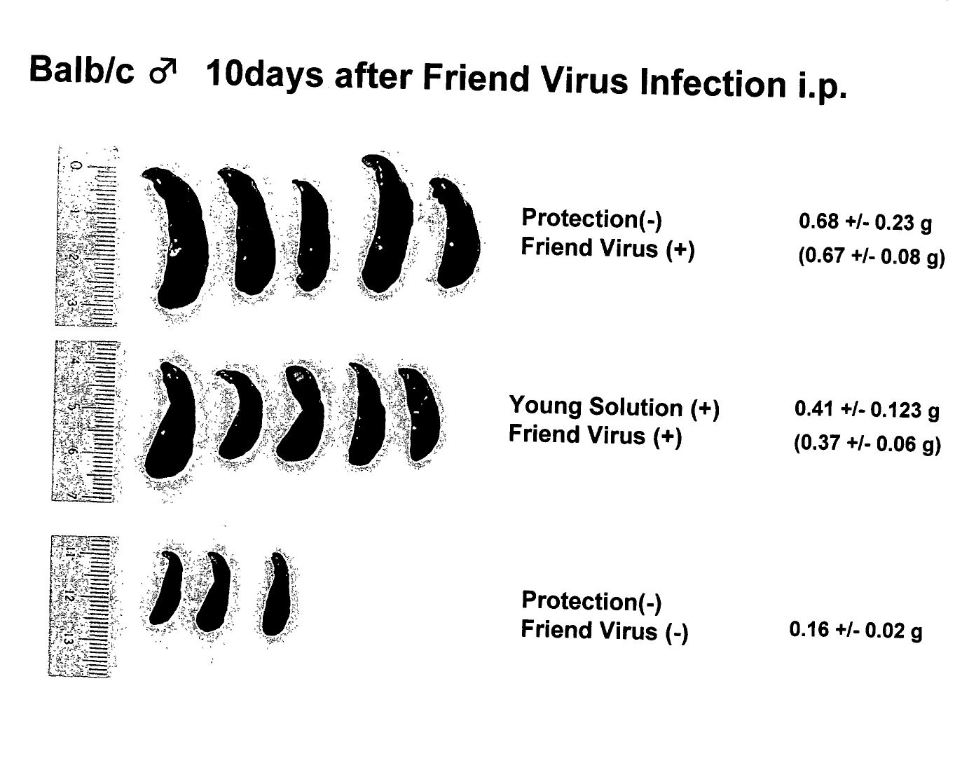

FIG. 1 shows a picture of the collected spleen of the test animals.

As shown, in contrast to animals in group 3, which were not subjected to Friend leukemia virus, enlarged spleen and development of leukemia were observed 10 days after infection in the animals in group 1 and group 2, which were each infected with Friend leukemia virus. In the untreated group (group 1), the weight of spleen was 0.676±0.225 g (0.666±0.081 g taken for n=3, discarding maximum and minimum values). In comparison, the weight of spleen was 0.405±0.123 g (0.367±0.058 g taken for n=3, discarding maximum and minimum values) in the treated group (group 2), showing a significant decrease equivalent to as much as 40% of the weight of affected spleen (46% taken for n=3, discarding maximum and minimum values). Consequently, it has proven that administration of the Young extract effectively delays the progress of the retroviral infection.

### Test 2

Test for Determining Effects of Young Extracts on Murine AIDS (MAIDS) Virus

Methods

Murine AIDS (MAIDS) virus was inoculated in the abdominal cavity of male C57BL6 mice (5 animals per one group) to allow AIDS to develop. The Young extract was diluted 100-fold with distilled water, and 100 ml of the extract solution was placed in a water supply bottle to allow the mice to ingest the extract solution. The solution was replaced with a fresh supply every other day. The amount of the solution ingested by each mouse was 5 ml/day/mouse on average.

The Young extract was administered over 3 consecutive months starting from one day prior to infection with murine AIDS (MAIDS) virus. The effects were determined 3 months after administration.

As a comparative experiment, a group of mice (5 animals) were inoculated with murine AIDS virus (MAIDS) but not administered with the Young extract. In a control group (3 animals), mice were not infected with murine AIDS virus (MAIDS), nor were they administered with the Young extract.

Results

In contrast to the untreated group (inoculated with murine AIDS virus (MAIDS) but not administered with the Young extract), lives of the mice were significantly extended in the treated group as with the control group.

As set forth, the present invention serves to induce secretion of biological anti-microbial peptides, an innate protective mechanism against viral infection, thereby bringing about significant anti-viral activity against retrovirus. Thus, the present invention provides a clinically effective therapeutic agent for treating retroviral infections, especially AIDS. The fermentation products produced by yeasts and lactic acid bacteria, the active ingredients of the therapeutic agent of the present invention, are highly safe substances and are free of adverse side effects. The products, therefore, are of significant medical importance.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A therapeutic agent for treating retroviral infection, comprising a concentrate of a filtrate of a culture solution obtained by co-culturing a particular combination of different types of beneficial bacteria selected from yeasts and lactic acid bacteria.

2. The therapeutic agent for treating retroviral infection according to claim 1, wherein the retroviral infection is acquired immune deficiency syndrome (AIDS) caused by human immunodeficiency virus (HIV).

3. The therapeutic agent for treating retroviral infection according to claim 1, wherein the retroviral infection is adult T-cell leukemia (ATL) caused by human T-cell lymphotropic virus-I (HTLV-I).

4. The therapeutic agent for treating retroviral infection according to claim 1, wherein the beneficial bacteria are selected from the group consisting of Bac. Bulgaricus, Bac. Koernchen, Bac. Acidophillus, Micrococcus Lactis, yeast, and mixtures thereof.

5. The therapeutic agent for treating retroviral infection according to claim 1, further comprising a pharmaceutically acceptable carrier.

6. A method of treating a mammal having a retroviral infection comprising administering the therapeutic agent of claim 1.

7. The method according to claim 6, wherein said therapeutic agent is administered orally.

8. The method according to claim 6, wherein the retroviral infection is acquired immune deficiency syndrome (AIDS) cause by human immunodeficiency virus (HIV).

9. The method according to claim 6, wherein the retroviral infection is adult T-cell leukemia (ATL) caused by human T-cell lymphotropic virus-I (HTLV-I).

10. A method of making the therapeutic agent of claim 1, comprising:
A. preparing a special agar culture medium by boiling beef and kelp in water to obtain, a broth and adding the broth to agar along with salt and a culture solution of the beneficial bacteria;
B. preparing pure cultures of beneficial bacteria by individually inoculating types of beneficial bacteria other than those in said culture solution onto said special agar culture medium and incubating each culture;
C. preparing a special soybean milk culture medium by boiling degreased soybean in water, filtering, and adding salt, brown sugar, and a culture solution of beneficial bacteria to the resulting filtrate;
D. preparing pure cultures of beneficial bacteria in special soybean milk culture medium by inoculating each pure culture of different types of beneficial bacteria prepared in step B onto the special soybean milk culture medium prepared in step C and incubating the resulting culture;
E. dividing the pure cultures of beneficial bacteria prepared in step D into different groups, inoculating each group onto the special soybean milk culture medium prepared in step C and acclimatizing;
F. inoculating each group of bacteria prepared in step E into special soybean milk culture obtained in step C and co-culturing the inoculated groups;
G. heating the co-culture solution from step F to terminate the growth of bacteria, removing bacterial cells, and concentrating the resulting filtrate; and
H. aging the concentrate obtained in step G.

11. A method according to claim 10, wherein the incubation in step B is performed for about 48 to 50 hours at about 39° C.

12. A method according to claim 10, wherein the boiling in step C is for about 70 to 110 minutes.

13. A method according to claim 10, wherein the incubation in step D is performed for about 48 to 50 hours at about 39° C.

14. A method according to claim 10, wherein the acclimation in step E is performed for about 48 to 50 hours at about 39° C.

15. A method according to claim 10, wherein each group of bacteria inoculated into about 13 L of special soybean milk culture as recited in step F and co-culturing for about 100-120 hours at about 40° C.

16. A method according to claim 10, wherein the heating in step G is for about 30 min at about 100° C., the bacterial cells are removed by filtering, the filtrate is concentrated about 15-fold.

17. A method according to claim 10, wherein the aging in step H is for about 3 months at about 170° C.
